SEQUENCE LISTING (9 sequences)

SEQ ID NO: 1 - protein, 540 aa, unknown
  Mycobacterium tuberculosis HSP60 (P0A521)
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATEVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDF

SEQ ID NO: 2 - protein, 16 aa, unknown
  M. tuberculosis HSP60 protein fragment
EGEALSTLVVNKIRGT

SEQ ID NO: 3 - protein, 101 aa, unknown [sequence omitted]

SEQ ID NO: 4 - protein, 339 aa, unknown
  Human HSP40 protein (P25685)
GKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 5 - protein, 331 aa, unknown
  Human HSP 40 protein (Q86TL9)
MVKETTYYDVLGVKPNATQEELKKAYRKLALKYHPDKNPNEGEKFKQISQAYEVLSDAKKRELYDKGGEQAIKEGGAGGGFGSPMDIFDMFFGGGGRMQRERRGKNVVHQLSVTLEDLYNGATRKLALQKNVICDKCEGRGGKKGAVECCPNCRGTGMQIRIHQIGPGMVQQIQSVCMECQGHGERISPKDRCKSCNGRKIVREKKILEVHIDKGMKDGQKITFHGEGDQEPGLEPGDIIIVLDQKDHAVFTRRGEDLFMCMDIQLVEALCGFQKPISTLDNRTIVITSHPGQIVKHGDIKCVLNEGMPIYRRPYEKGRLIIEFKVSCNVL

SEQ ID NO: 6 - protein, 573 aa, unknown
  Human HSP60 protein (P10809)
  Signal peptide (1)..(26)
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 7 - protein, 654 aa, unknown
  Human HSP70 protein (P11021)
  signal peptide (1)..(18)
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 8 - protein, 723 aa, unknown
  Human HSP90 protein (P08238)
PEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 9 - protein, 633 aa, unknown
  Human HSP100 protein (O76031)
  signal peptide (1)..(56)
MPSCGACTCGAAAVRLITSSLASAQRGISGGRIHMSVLGRLGTFETQILQRAPLRSFTETPAYFASKDGISKDGSGDGNKKSASEGSSKKSGSGNSGKGGNQLRCPKCGDLCTHVETFVSSTRFVKCEKCHHFFVVLSEADSKKSIIKEPESAAEAVKLAFQQKPPPPPKKIYNYLDKYVVGQSFAKKVLSVAVYNHYKRIYNNIPANLRQQAEVEKQTSLTPRELEIRRREDEYRFTKLLQIAGISPHGNALGASMQQQVNQQIPQEKRGGEVLDSSHDDIKLEKSNILLLGPTGSGKTLLAQTLAKCLDVPFAICDCTTLTQAGYVGEDIESVIAKLLQDANYNVEKAQQGIVFLDEVDKIGSVPGIHQLRDVGGEGVQQGLLKLLEGTIVNVPEKNSRKLRGETVQVDTTNILFVASGAFNGLDRIISRRKNEKYLGFGTPSNLGKGRRAAAAADLANRSGESNTHQDIEEKDRLLRHVEARDLIEFGMIPEFVGRLPVVVPLHSLDEKTLVQILTEPRNAVIPQYQALFSMDKCELNVTEDALKAIARLALERKTGARGLRSIMEKLLLEPMFEVPNSDIVCVEVDKEVVEGKKEPGYIRAPTKESSEEEYDSGVEEEGWPRQADAANS

(19) United States
(12) Patent Application Publication
(10) Pub. No.: US 2008/0039400 A1
(43) Pub. Date: Feb. 14, 2008

(54) TREATMENT AND PREVENTION OF INFLAMMATORY BOWEL DISEASES

(75) Inventors: Willem VAN EDEN, Bilthoven (NL); Ruurd Van Der Zee, Utrecht (NL); Helena Tlaskalova-Hogenova, Prague (CZ); Miloslav Kverka, Prague (CZ)

Correspondence Address: FOLEY AND LARDNER LLP;SUITE 500, 3000 K STREET NW, WASHINGTON, DC, 20007, US

(73) Assignee: Universiteit Utrecht UU Holding B.V.

(21) Appl. No.: 11/626,152

(22) Filed: Jan. 23, 2007

Related U.S. Application Data
(60) Provisional application No. 60/761,339, filed on Jan. 24, 2006.

(51) Int. Cl. A61K 38/00; A61P 1/00

(52) U.S. Cl. 514/13; 514/14; 514/15; 514/16; 514/17

(57) ABSTRACT

The present invention relates to the use of heat shock proteins, or fragments thereof, for the treatment and prevention of Inflammatory Bowel Diseases. Preferably bacterial and/or human heat shock proteins, especially those belonging to the HSP60 family, are used.

### CROSS-REFERENCE TO RELATED PATENT APPLICATIONS

This patent application claims the benefit under 35 U.S.C. §119(e) of U.S. Provisional Application No. 60/761,339, filed Jan. 24, 2006, which is herein incorporated by reference in its entirety.

### FIELD OF THE INVENTION

The present invention relates to the treatment and/or prevention of Inflammatory Bowel Diseases (IBD), including Crohn's disease, ulcerative colitis, lymphocyte colitis, collagenous colitis and/or Coeliac disease. Especially, the use of bacterial and/or human heat shock proteins (HSPs), or fragments thereof, for the preparation of compositions for the treatment or prophylaxis of one or more IBDs is provided, as are methods for therapeutic or prophylactic treatment of one or more IBDs. In one embodiment of the invention, methods and compositions for the treatment and/or prevention of Lymphocyte colitis, collagenous colitis and/or Coeliac disease are provided.

### BACKGROUND OF THE INVENTION

Heat shock proteins (HSP) have shown to be critical to type 1 diabetes mellitus and rheumatoid arthritis, both of which are prevalent chronic degenerative autoimmune diseases. The criticality was based on the following findings:
1. Peptides of HSP's can be used as therapeutic agents to prevent or arrest the inflammatory damage in both experimental autoimmune arthritis and in experimental autoimmune diabetes. The peptide treatments (in first clinical trials) are marked by a shift in the cytokine profiles of specific autoimmune T cells from a pro-inflammatory Th-1 response to an anti-inflammatory Th-2 response.
2. Epitopes of HSP's are recognized by the adaptive arm of the immune system (antigen receptors of T cells and B cells)
3. Epitopes of HSP's are targets for regulatory T-cells in both diseases.

In models of type I diabetes and arthritis, immunization with HSP has been seen to prevent and to suppress disease. The probable mechanism here is the expansion of microbial (commensal) HSP reactive T cells, tolerised in the gut through mechanisms of mucosal tolerance. This expansion of HSP reactive T cells was possible through both oral and parenteral routes of HSP administration. The expanded T cells are cross-reactive with homologous self-HSP over-expressed in the inflamed (stressed) tissue. And this cross-reactivity of tolerant T cells does lead to regulatory cytokine production at the site of inflammation. For type I diabetes and arthritis first clinical trials in humans have shown the potential of HSP derived peptides to switch cytokine patterns of disease associated T cell specificities into more regulatory cytokine production.

WO 03/054011 describes a method of determining susceptibility to inflammatory bowel disease (IBD) using polynucleotide probes corresponding to the Organic Cation Transporter (OCTN) genes.

WO 2005/048914 discloses DNA vaccines encoding a human heat shock protein hsp60 fragment for preventing or treating inflammatory autoimmune diseases. The hsp60 fragments are identified by their reaction with T cells previously sensitized to human hsp70. The autoimmune diseases listed also mention IBD amongst many other diseases. However, IBD is not an autoimmune disease, and the experiments presented are restricted to the autoimmune disease arthritis without any evidence of an activity against IBD.

WO 2005/022160 describes a method of diagnosing paratuberculosis in ruminants by administering a heat shock protein, in particular hsp70, or part thereof, of the causative agent Mycobacterium avium paratuberculosis and detecting the immune response to the protein. The immune response to the protein is also suitable for treating or preventing paratuberculosis infection.

WO95/25744 describes the use of parts of mycobacterial heat shock proteins having mammalian sequence similarity for protection against or treatment of an inflammatory disease, including autoimmune diseases, such as diabetes, arthritic diseases, atherosclerosis, multiple sclerosis, myasthenia gravis, or inflammatory responses due to tumour or transplant rejection. The document is silent about IBD.

Hutszti, Bene et al. (2004, Inflamm Res 53: 551-555) describe experiments aimed at supporting the observation that low levels of antibodies against mycobacterial hsp65 are found in patients with IBD. There is no indication that bacterial hsp60 proteins or human heat shock proteins can be used to treat or prevent IBD.

Elsaghier et al. (1992, Clin. Exp. Immunology 89: 305-309) describe the measurement of antibody levels to mycobacterial and human heat shock proteins in patients with Crohn's disease, ulcerative colitis and non-tuberculous mycobacterial diseases of the lung. They conclude that the data are not sufficient to imply sensitization with mycobacteria in patients with IBD. Thus, other bacterial proteins may be involved in sensitization and there is no indication that bacterial hsp60 proteins or human heat shock proteins can be used to treat or prevent IBD.

The origin of inflammatory bowel diseases (IBD) is known to depend on the presence of bacterial gut flora and is regarded as an inappropriate hyper-responsiveness to commensal organisms (Bouma and Strober 2003, Nature Rev. Immunol. 3: 521-533). In surgically excluded ileum of Crohn's patients (no fecal stream) lesions were seen to disappear. Infusion of intestinal contents induces recurrent Crohn's disease (D'Haens et al. 1998, Gastroenterology 114:262-267). Moreover, under germ-free conditions no experimental IBD disease can be induced, unless the gut flora is reconstituted (Chandran et al. 2003, Surgeon 1:125-136, Strober et al. 2002, Annu. Rev. Immunol 20:495-549). Therefore, supposedly, bacterial antigens are the trigger leading to the induction of disease. In IBD no causally related auto-antigens are known to exist, which is in contrast to auto-immune diseases. IBD are, therefore, considered not to be auto-immune diseases, such as type 1 diabetes and rheumatoid arthritis.

Models of IBD have generated evidence for a primary role of anaerobic bacteria (Clostridium, Bacteroides) in the induction of disease (see Verdu et al. 2000, Clin Exp Immunol. 120(1):46-50). Crude sonicates of anaerobic, aerobic gram positive and gram negative bacteria have been administered orally in DSS induced colitis and only sonicates of anaerobic bacteria were found to reduce severity of experimental colitis (Verdu et al. 2000, Clin Exp Immunol. 120(1):46-50).

### SUMMARY OF THE INVENTION

The present inventors tested recombinant mycobacterial HSP60 (both a full length protein, as shown in SEQ ID NO: 1 (P0A521) and a 16 amino acid fragment thereof shown in SEQ ID NO: 2) by oral administration in the rodent DSS model of colitis and found it to be highly disease suppressive, both when administered prophylactically (prior to onset of disease) and if administered therapeutically (after onset of disease). The results were completely unexpected and contrary to what had been observed previously, because intraperitoneally injected Yersinia-derived HSP60 had been seen to induce colitis and HSP60 reactive CD8 T cells had been seen to cause intestinal inflammation in earlier studies (Yagita et al., 1999, Dig Dis Sci., 44(2):445-51; Steinhoff et al., 1999, Immunity 11(3):349-58).

Provided is the use of a bacterial and/or of a mammalian (especially a human) heat shock protein, or a fragment of at least 6 or 7 (or more) contiguous amino acids thereof, for preparing a medicament for the treatment or prevention of inflammatory bowel diseases (IBD). When referring to IBD herein, it is understood that also reference to one or more IBDs is referred to, such as specifically Lymphocyte colitis, collagenous colitis and/or Coeliac disease.

Bacterial heat shock proteins suitable for use against (one or more) IBDs preferably comprise at least 35% amino acid sequence identity over the entire length to a mammalian heat shock protein of the same heat shock protein family, selected from HSP10, HSP40, HSP60, HSP70, HSP90 and HSP100.

A human heat shock protein suitable for use against IBD preferably comprises at least 35 or 40% amino acid sequence identity over the entire length of the protein to a human heat shock protein of the same heat shock protein family, selected from HSP10, HSP40, HSP60, HSP70, HSP90 and HSP100, such as depicted in SEQ ID NO: 3-9.

In one embodiment the HSP used belongs to the HSP60 family, and in a preferred embodiment the protein is a full length protein, preferably the protein of SEQ ID NO: 1 or the protein consists of or comprises the amino acid sequence of SEQ ID NO: 2. In another preferred embodiment the protein is SEQ ID NO: 6 (human HSP60), or a variant thereof, or a fragment of any of these.

Also a method for the treatment or prevention of inflammatory bowel diseases (IBD) in a human is provided, comprising administering to a person in need thereof a therapeutically or prophylactically effective amount of a bacterial or of a human heat shock protein, or fragment of at least 6 or 7 (or more, e.g. 15 or 16) contiguous amino acids thereof.

General Definitions

“IBD” refers herein to Inflammatory Bowel Diseases, a chronic inflammation of the gastrointestinal tract, comprising the following diseases: Crohn's disease, ulcerative colitis, lymphocyte colitis, collagenous colitis and/or Coeliac disease. “Autoimmune diseases” refers to diseases such as insulin-dependent diabetes mellitus (type 1 diabetes), artherosclerosis, myasthenia gravis, experimental autoimmune encephalomyelitis, etc. wherein the primary disease initiating and maintaining immune response is directed against auto-antigens (self antigens; i.e. antigens of normal cellular components) of the subject. Autoantibodies and T cells are produced, which are specific for such autoantigens.

“Non autoimmune diseases” refers herein to diseases wherein the primary disease initiating and maintaining immune response is not directed against auto-antigens (self antigens) of the subject, but against non-self antigens (foreign antigens). For example, IBDs are non autoimmune diseases. In this/the latter case autoimmune responses are not responsible for initiating and/or maintaining the inflammation. In the case of Crohn's disease the non-autoimmune nature of the disease was demonstrated in surgically constructed blind-loops, where removal of fecal content led to resolution of the disease, whereas re-infusion of fecal contents led to disease recurrences (Rutgeerts et al. 1991, Lancet 338:771-774).

“Subject(s)” are herein mammals, especially humans.

The term “antigen” (or immunogen) includes reference to a substance capable of eliciting an adaptive immune response, i.e. to induce production of antigen recognition molecules (especially antigen specific or cross-reactive T cells) to which the antigen is specifically immunoreactive. The specific immunoreactive sites within the antigen are known as “epitopes” (or antigenic determinants). Herein proteins or protein fragments (peptides) consisting of, or comprising, one or more epitopes of bacterial HSP proteins capable of preventing or treating (one or more) IBDs are provided. These epitopes are also referred to as “protective epitopes”.

“T cell epitope” refers to the epitopes recognized by the T cell receptors. Upon binding of the epitope, an immune response is mounted in the subject.

“Enteral” refers herein to the delivery directly into the gastrointestinal tract of a subject (e.g. orally or via a tube, catheter or stoma).

“Percentage” or “average” generally refers to percentages of averages by weight, unless otherwise specified or unless it is clear that another basis is meant.

“Sequence identity” and “sequence similarity” can be determined by alignment of two amino acid sequences or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimizes the number of gaps. For sequences of equal length or approximately equal length, a global alignment algorithm is preferred (while for sequences of dissimilar length a local alignment algorithm is preferred, such as Smith-Waterman). Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Also, EmbossWin version 2.10.0 can be used, using the program ‘needle’ (which corresponds to GAP) with the same parameters as for GAP above. Alternatively percent similarity or identity may be determined by searching against databases, using algorithms such as FASTA, BLAST, etc.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial host cell.

Depending on the context, the term “homolog” or “homologous” refers to sequences which are descendent from a common ancestral sequence. If desired, the term can be specified by referring to orthologs and paralogs, see http://www.ncbi.nlm.nih.gov/Education/BLASTinfo/Orthology.html). Orthologs generally retain the same function in a different species. Paralogs, in contrast evolved different (possibly related) functions. Bacterial heat shock proteins and mammalian heat shock proteins of the corresponding HSP family are, thus, herein referred to as homologs.

The terms “homologous” and “heterologous” may also be used to refer to the relationship between a nucleic acid or amino acid sequence and its host cell or organism, especially in the context of transgenic cells/organisms. A homologous sequence is thus naturally found in the host species, while a heterologous sequence is not naturally found in the host cell.

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions. Stringent conditions for DNA-DNA hybridization (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a cell” refers also to several cells in the form of cell cultures, tissues, whole organism, etc. It is further understood that, when referring to “sequences” herein, generally the actual physical molecules with a certain sequence of subunits (e.g. amino acids) are referred to.

### DETAILED DESCRIPTION

The invention pertains to the use of bacterial and/or human heat shock proteins, or fragments thereof, for the treatment and/or prevention of IBD, especially various forms of colitis, such as those selected from the group consisting of Crohn's disease, ulcerative colitis, lymphocyte colitis, collagenous colitis and/or Coeliac disease.

Proteins and Peptides for Use According to the Invention

Heat shock proteins are universal proteins, which carry out important house-keeping functions of prokaryotic and eukaryotic cells. They play an important role as chaperones in protein folding and in rescuing the cell from stress conditions. They are classified into different families on the basis of their monomeric molecular weight. Thus proteins of the family HSP60 have a molecular weight of about 60 kDa (and include for example the Mycobacterial HSP65 protein). The main families are HSP 10, 40, 60, 70, 90 and 100. Many mammalian HSP family members have highly conserved microbial homologs. For example, the Mycobacterial HSP65 protein shares about 48% amino acid identity with the mammalian homolog (HSP60 or P1). Karlin and Brocchieri (PNAS 2000, vol. 97, page 11348-11353) compare members of the HSP60 family. Also FIG. 13 of EP0751957 shows a multiple sequence alignment between human, rat, mouse and mycobacterial HSP60 family members, which share 44.3% sequence identity over a consensus sequence of 540 amino acids.

HSP60 (also referred to as GroEL or HSP65 in bacteria) belongs to the HSP60 family and is present in all prokaryotes and not specifically in anaerobic bacteria only. HSP60 homologs are also present in eukaryotic cells, such as for example in the mitochondria or plastid organelles of eukaryotic cells. For example, pairwise sequence alignments of the mature, processed proteins (plastid transit peptides removed, where present in the eukaryotic proteins) provides the following percentage sequence identity between mammalian (human, rat and mouse) and Mycobacterial HSP60 family proteins.

| | | Rat HSP60 | Mouse HSP60 | |
|---|---|---|---|---|
| | Human HSP60 | (CPN60; | (CPN60; | Mycobacterium |
| | (P1 or CPN60) | HSP65) | HSP65) | bovis HSP65 |
| | (P10809) | (P63039) | (P63038) | (P0A521)* |
| Rat HSP60 | 97.8% | 100 | | |
| (P63039) | | | | |
| Mouse HSP60 | 97.8% | 100% | 100 | |
| (P63038) | | | | |
| M. bovis | 46.6% | 47.1% | 47.1% | 100 |
| HSP65 | | | | |
| (P0A521)* | | | | |

*corresponds to the SEQ ID NO: 1

Percentage sequence identity was calculated using EMBOSSwin—v2.10.0, “needle”, GAP creation penalty=8.0 and GAP extension penalty=2.0. Alternative names and NCBI/Swiss-Prot accession numbers of the sequences are indicated. The mitochondrial transit peptides of the human, mouse and rat sequences were excluded (amino acids 1-26).

The present inventors tested recombinant mycobacterial HSP60 (both a full length protein, as shown in SEQ ID NO: 1 and a fragment thereof (shown in SEQ ID NO: 2) by oral administration in the rodent DSS model of colitis and found it to be highly disease suppressive (see Examples). Both a prophylactic effect as well as a treatment effect was observed.

Thus, in one embodiment protective epitopes, or proteins or protein fragments consisting of or comprising protective epitopes are provided. Especially in one embodiment bacterial HSP proteins, or fragments thereof, are used to prepare compositions (or medicaments) for the treatment and/or prophylaxis of one or more IBDs. In principle, any bacterial HSP protein, or fragment thereof, may be used, such as any HSP protein of a gram positive or gram negative bacterial species. Preferably the bacterial HSP belongs to a family of the group: HSP10, HSP40, HSP60, HSP70, HSP90 and HSP100. Especially preferred are bacterial HSP60 proteins, and most preferred is the HSP60 protein of Mycobacterium species, such as the HSP60 of Mycobacterium tuberculosis (same sequence as HSP60 of M. bovis BCG) depicted in (SEQ ID NO: 1).

In another embodiment of the invention a mammalian, especially a human HSP (preferably HSP60, such as human HSP 60 shown in SEQ ID NO: 6), a variant or fragment thereof is used, as described elsewhere herein.

When referring to “a bacterial heat shock protein” it is understood that the protein occurs in bacteria in nature, but may be produced or isolated by various means. For example, it may be produced by recombinant DNA technology, whereby the nucleotide sequence encoding the protein (or protein fragment) is used to transform or transfect a host cell, which then produces the protein or protein fragment. Nucleic acid sequences (cDNA, RNA and genomic DNA) encoding bacterial HSPs are available in the art or may be made by chemical synthesis, and the methods for recombinant production of the protein or protein fragment (peptide) are routine. Similarly, nucleic acid hybridization techniques (for example using stringent hybridization conditions) may be used to isolate genes encoding bacterial HSPs. Alternatively, the protein or protein fragment may be purified or partially purified from natural sources (e.g. the natural bacteria) or may be synthesized chemically. For example, the peptides can be synthesized by the well-known Merrifield solid-phase synthesis method in which amino acids are sequentially added to a growing chain. See Merrifield (1963), J. Am. Chem. Soc. 85:2149-2156; and Atherton et al., “Solid Phase Peptide Synthesis,” IRL Press, London, (1989). Automatic peptide synthesizers are commercially available from numerous suppliers, such as Applied Biosystems, Foster City, Calif.

In the broadest sense, any (bacterial) HSP protein capable of inducing cross-reactive T cell responses to human HSP (including human HSP itself).

Although the protein may be derived from any bacterium, it is preferably derived from a bacterium belonging to a genus selected from the group consisting of: Mycobacterium (e.g. M. tuberculosis, M marinum, M. ulcerans, M. avium, etc.), Norcadia, Nocardioides, Cornybacterium, Arthorbacter, Propionibacterium, Brevibacterium, Kineococcus, Streptomyces, Thermobifida, Bifidobacterium, Rubrobacter, Synechococcus, Heliobacillus, Clostridium, Bacillus, Staphylococcus, Streptococcus, Enterococcus, Coxellia, Mesorhizobium, Lactobacillus, Lactococcus, and others.

As mentioned above, “derived from” does not imply that the protein or fragment thereof must have been isolated from the bacterium, but it may equally be synthesized, produced by recombinant means, etc.

Bacterial heat shock proteins according to the invention comprise all bacterial HSP proteins having at least 35%, 40%, 45%, 50%, 60%, 70%, 80%, 90%, 95%, 98%, 99% or more (100%) amino acid sequence identity (over the full length of the protein) to mammalian (e.g. rodent, such as mouse or rat, or human) heat shock proteins of the same HSP family (also referred to as “variants” of bacterial HSPs), most preferably to human heat shock proteins of the same family, as well as functional fragments of any of these. To determine whether a protein has the specified percentage sequence identity the two full length amino acid sequence are aligned using the Needleman-Wunsch algorithm (e.g. the program “GAP”, or the program “needle” in EmbossWin version 2.10.0) using a gap creation penalty of 8 and a gap extension penalty of 2. The plastid transit peptide (e.g. mitochondrial transit peptide) of the mammalian HSP is removed prior to the alignment. “Full length” or “entire length” thus refers to the full length bacterial sequence and the processed (mature) mammalian sequence, whereby any putative signal peptide at the N-terminal of the protein is removed.

The “of same HSP family” refers to homologs or orthologs of HSP proteins (and genes encoding these) found in different species, but which are grouped into the same major HSP family, selected from HSP10, HSP40, HSP60, HSP70, HSP90 and HSP100. For example, known members of the HSP60 family include those referred to under various synonyms, such as GroE1, Cpn60, Hsp60, 60 kDa chaperonin, Heat shock protein 60, HSP-60, Mitochondrial matrix protein P1, P60 lymphocyte protein, HuCHA60, Protein Cpn60-2, groEL protein 2, 65 kDa antigen, Heat shock protein 65, Cell wall protein A, etc. The more generic terms used are HSP60 and CPN60.

“Fragments” refer herein to peptides comprising at least 6 or 7, more preferably at least 8 or 9, more preferably 10, 11, 12, 13, 14, 15, 16, 17, 18, 20, 25, 30, 40, 50, 55, 60, 70 or more consecutive (contiguous) amino acids of any of the HSPs according to the invention.

Both full length HSP proteins and fragments according to the invention are preferably “functional”, i.e. they consist of or comprise one or more protective epitopes and are capable of inducing the production of cross-reactive T-cells when administered to a subject and, especially, to reduce or prevent IBD or one or more symptoms associated with IBD. Thus, in the broadest sense, any HSP protein (or protein fragment) capable of inducing cross-reactive T cell responses to human HSP (including human HSP itself) is suitable for use.

Various tests for functionality of the full length proteins or protein fragments can be applied. For example, the determination of T cell epitope qualities of selected sequences can be carried out as follows:
a) After immunizations with the whole bacterial HSP protein, T cell responses to the peptides are monitored, to determine whether individual T cell epitopes are contained within the selected peptides. Additionally, since immunity to conserved bacterial proteins is likely to be pre-existent, due to priming with bacterial antigens, epitopes can be detected by direct in vitro screening of secondary T cell responses to the peptides including the mammalian/human HSP peptides themselves, without prior immunization.
b) Alternatively, by immunizations with these peptides their capacity to induce sequence specific T cell responses to the microbial HSP protein and to the mammalian/human HSP protein, will reveal their T cell epitope qualities.

A priori prediction of possible epitopes can be made on the basis of known MHC (HLA) binding motifs. The in vitro method for screening in all cases can be performed by the use of standard lymphocyte proliferation assays. Alternatively, other signs of T cell activation can be measured, such as production of cytokines, Ca2+ fluxes, cell body enlargement and increased or changed cell surface marker expression. Definition of T cell epitopes can be done in patients, healthy individuals and/or vaccinated/specifically immunized individuals. These individuals are preferably HLA-typed.

Tests for determination of the capacity of selected epitopes to induce T cells cross-reactive with homologous self-proteins include: T cells activated in vitro with the defined microbial epitopes, can then be restimulated with the homologous self protein (in the rat model the rat HSP, either as a recombinant protein or purified from stressed cells or tissue or as elevated levels of MHC-peptide complexes on stressed antigen presenting cells) or the homologous peptide. Any sign of activation (see above) can be taken as an indication of cross-reactivity of the microbial epitope with the self protein. Initial testing can be carried out with a synthetic peptide based on the homologous sequence of the self protein, but final proof for cross-reactivity with the protein itself, either in isolated form or expressed on cells, should be obtained, in order to exclude cryptic epitopes.

An easy way of determining the most suitable fragment is to generate overlapping peptides (for example overlapping pentamers, hexamers, heptamers, or decamers; i.e. short consecutive amino acids) of a full length bacterial HSP protein or mammalian/human HSP protein and to screen these overlapping peptides for their protective effect. For example, by administration in a rodent model of Colitis, as described in the Examples.

Alternatively, an algorithm may be used to predict which fragments are most suitable and to make a selection based on this predicted protective effect. For example, protein regions comprising predicted mammalian (e.g. human, mouse or rat) HSP60 T-cell epitopes may be found (or predicted a priori based on known MHC (HLA) binding motifs) and based on this information bacterial fragments (or whole proteins) comprising or consisting of the predicted mammalian HSP60 T cell epitope, may be used. Likewise the predicted mammalian/human epitope fragments may be used.

In a preferred embodiment the complete HSP protein is used. For example the mycobacterial HSP60 (SEQ ID NO: 1) consists of 540 amino acids in length. In another preferred embodiment a fragment comprising or consisting of at least about 6, 7, 8 or more consecutive amino acids of SEQ ID NO: 1 is used, for example SEQ ID NO: 2.

Examples of HSP proteins (and variants and fragments thereof as defined above) suitable for use according to the invention include bacterial proteins comprising the above percentage amino acid sequence identity to any one of the following sequences:
Human HSP10-SEQ ID NO: 3 (SwissProt P61604);
Human HSP40-SEQ ID NO: 4 (SwissProt P25685);
Human HSP40-SEQ ID NO: 5 (SwissProt Q86TL9)
Human HSP60-SEQ ID NO: 6 (SwissProt P10809);
Human HSP70-SEQ ID NO: 7 (SwissProt P11021);
Human HSP90-SEQ ID NO: 8 (SwissProt P08238);
Human HSP100-SEQ ID NO: 9 (SwissProt O76031).

The signal peptide sequence, as indicated in the sequence listing, is excluded when calculating percentage amino acid identity.

In another embodiment according to the invention, HSP proteins for use according to the invention comprise all proteins having at least 35%, 40%, 45%, 50%, 60%, 70%, 80%, 90%, 95%, 98%, 99% or more (100%) amino acid sequence identity (over the full length of the protein) to any one of SEQ ID NO: 1-9 (also referred to as “variants” of SEQ ID NO: 1-9), or one or more (functional) fragments thereof (as defined above), irrespective of the origin of the protein and irrespective of whether it occurs naturally (in nature).

In these embodiments, it is not required that the protein occurs naturally in bacteria or mammals (e.g. humans), as herein variants of the naturally occurring HSP protein are used. Such variants may, off course, also occur naturally in bacteria or mammals. These variants (and fragments thereof) may be generated by methods known in the art, such as site directed mutagenesis, de novo chemical synthesis, recombinant expression of nucleic acid sequences comprising deletions, replacements, or additions of one or more nucleotides, gene shuffling techniques, etc. For example small modifications to a DNA sequence such as described above can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Preferred fragments of the Mycobacterial HSP60 (as depicted in SEQ ID NO: 1) consist of or comprise the following amino acids: amino acid 253-268 of SEQ ID NO 1 (which is also depicted as SEQ ID NO: 2), or any other fragment of at least 6, 7, 8, 9, 10, 11, 12, 13, 14, 15, 16, 17, 18, 20, 30, 40, 50 or more, consecutive amino acids of SEQ ID NO: 1. Most preferred are fragments which are highly conserved among hsp60 proteins. Non-limiting examples of fragments include sequences consisting of or comprising amino acids: 22-26, 31-35, 84-95, 171-175, 247-252, 254-261, 272-281, 365-370 and/or 403-410 of SEQ ID NO: 1.

In one embodiment it is preferred that only one protein or protein fragment is used, while in another embodiment mixtures of proteins and/or fragments of different amino acid sequence may be used. Thus, for example the whole mycobacterial HSP60 protein may be mixed with one or more fragments of the mycobacterial HSP60 protein. Alternatively, proteins and/or fragments of bacterial HSP proteins of different HSP families may be mixed. Also bacterial and human HSP proteins, or functional fragments thereof may be mixed. Equally mixtures of human HSP proteins or fragments, of the same or different HSP family, may be mixed.

In yet another embodiment of the invention protective epitopes, or proteins or protein fragments consisting of or comprising protective epitopes are provided, whereby human HSP proteins, or variants thereof, or fragments of any of these, are used to prepare compositions (or medicaments) for the treatment and/or prophylaxis of IBDs. “Human heat shock proteins” refer to proteins occurring naturally in Homo sapiens, such as those depicted in SEQ ID NO 3-9. Preferably, the heat shock protein used is the HSP60 protein of humans, such as the HSP60 depicted in (SEQ ID NO: 6), or a variant, or (functional) fragment of SEQ ID NO: 6 or of a variant of SEQ ID NO: 6. Throughout the description, the embodiments described for bacterial heat shock proteins apply equally to human heat shock proteins, especially human HSP60, as well as variants and fragments thereof.

Human heat shock proteins according to the invention comprise all human HSP proteins having at least 35%, 40%, 45%, 50%, 60%, 70%, 80%, 90%, 95%, 98%, 99% or more amino acid sequence identity (over the full length of the protein) to a human heat shock protein of the same HSP family (referred to as “variants” of human HSPs), for example to the amino acid sequence depicted in SEQ ID NO: 3-9. Also encompassed are fragments of any of these, especially functional fragments. “Fragments” refer herein to peptides comprising at least 6, 7 or 8, more preferably 9, 10, 11, 12, 13, 14, 15, 16, 17, 18, 19, 20, 30, 40, 50, 60, 70, or more consecutive (contiguous) amino acids of any of the human HSPs or variant according to the invention. Again, both the whole protein or the fragments used are preferably “functional”, i.e. they consist of or comprise one or more protective epitopes and are capable of inducing the production of cross-reactive T-cells when administered to a subject and, especially, to reduce or prevent IBD or one or more symptoms associated with IBD. Thus, to test functionality of a full length protein or fragment, the activation of cross-reactive T cells is measured as described above or as in the Examples.

It is understood that the invention also concerns peptide analogues, or proteins or proteins fragment comprising peptide analogues, which exhibit the immunological properties of the peptides described above, but which contain one or more chemical modifications. Such peptide analogues, also referred to as peptide mimetics, can e.g. consist of units corresponding to the amino acid residues of the peptides described above, wherein essentially the same side groups are present, but wherein the backbone contains modifications such as substitution of an amide group (CO—NH) by another group such as CH═CH, CO—O, CO—CH₂ or CH₂—CH₂. Other modifications, such as substitutions of an amino acid by a similar natural, or non-natural amino acid are also envisaged. In this respect, “similar” means having about the same size, charge and polarity; thus the aliphatic amino acids alanine, valine, norvaline, leucine, isoleucine, norleucine and methionine can be considered as similar; likewise the basic to neutral polar amino acids such as lysine, arginine, ornithine, citrulline, asparagine and glutamine are similar for the present purpose; the same applies to the acidic to neutral polar aminoacids like asparagine, aspartate, glutamine, glutamate, serine, homoserine and threonine.

The peptides described above may be used as such, or may be coupled to a sequence which enhances their antigenicity or immunogenicity. Such sequences may include parts of toxoids or immunoglobulins. The peptides may also be used as complexes with MHC molecules and/or incorporated in liposomes. The peptides may also be covalently coupled to other molecules or whole cells as a vector for immunostimulation. The peptides may be in the form of monomers, dimers or multimers.

The invention also provides autologous T cells or other cells expressing a T cell receptor, or part thereof, from such T cells, activated by immunostimulation using a protein and/or peptide as described above.

The invention also concerns antibodies, in particular monoclonal antibodies directed at the protein and/or peptides described above. The antibodies can be produced using known methods, e.g. by hybridoma technology. The antibodies may be used as a passive vaccine or as a diagnostic tool.

Uses and Methods According to the Invention

The proteins and/or peptides of the present invention are used to make pharmaceutical compositions comprising these, whereby the pharmaceutical compositions are useful for administration to mammals, particularly humans, to treat and/or prevent IBD (especially Crohn's disease, ulcerative colitis, lymphocyte colitis, collagenous colitis and/or Coeliac disease), or one or more (preferably all) symptoms thereof.

The amount of protein or peptide to be used may vary, depending on whether the composition is for the treatment or for the prophylaxis, and depending on the dosage form and frequency of administration. Suitable formulations are found in Remington's Pharmaceutical Sciences, Mack Publishing Company, Philadelphia, Pa., 17th ed. (1985), which is incorporated herein by reference.

In one embodiment the immunogenic proteins and/or peptides of the invention (or compositions comprising these) are administered prophylactically (prevention) or to an individual already suffering from IBD (treatment). The compositions are administered to a patient in an amount sufficient to elicit an effective immune response. An amount adequate to accomplish this is defined as “therapeutically effective dose” or “immunogenically effective dose.” Amounts effective for this use will depend on, e.g., the protein and/or peptide composition, the manner of administration, the stage and severity of the disease being treated, the weight and general state of health of the patient, and the judgement of the prescribing physician, but generally range for the from about 0.1 μg to about 150 μg per kilogram (kg) of body weight per patient, more commonly from about 1 μg to about 50 μg per kg of body weight per dose. A dose may be administered once a week, or once every other day or daily or even several times per day. Dosage units may be administered over a short period (e.g. a few weeks to months) or over longer time periods (several months to years).

The composition may be made in various dosage units, such as doses comprising e.g. 7 μg, 7.5 μg, 8 μg, 9 μg, 10 μg, 20 μg, 30 μg, 40 μg, 50 μg, 100 μg, 1000 μg, 2500 μg, 5000 μg or more protein and/or peptide.

Preferably, the pharmaceutical compositions are administered enteral, most preferably oral. However, in another embodiment other forms of administration are included, such as transdermal, nasal, inhalation, and parenteral.

The proteins and/or peptides according to the invention may, for example, be dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration.

The compositions may contain pharmaceutically acceptable auxiliary substances as required to approximate physiological conditions, such as buffering agents, tonicity adjusting agents, wetting agents and the like, for example, sodium acetate, sodium lactate, sodium bicarbonate, sodium chloride, potassium chloride, calcium chloride, sorbitan monolaurate, and triethanolamine oleate.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more proteins and/or peptides of the invention, and more preferably at a concentration of 25%-75%. As noted above, the compositions are intended to induce an immune response to the peptides. Thus, compositions and methods of administration suitable for maximizing the immune response are preferred. For instance, peptides may be introduced into a host, including humans, linked to a carrier or as a homopolymer or heteropolymer of active peptide units. Alternatively, the a “cocktail” of proteins and/or peptides can be used. A mixture of more than one protein and/or peptide has the advantage of increased immunological reaction and, where different peptides are used to make up the polymer, the additional ability to induce antibodies to a number of epitopes. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like.

The compositions, especially oral dosage forms, may further comprise one or more protease inhibitors. Protease inhibitors are devided into four classes: serine protease inhibitors (including trypsin inhibitors), cysteine protease inhibitors, aspartic protease inhibitors, and metalloproteinase inhibitors. Suitable protease inhibitors are available in the art (e.g. from Sigma-Aldrich). A preferred inhibitor is a trypsin inhibitor, such as a plant derived trypsin inhibitor (soybean trypsin inhibitor, lima bean trypsin inhibitor, corn trypsin inhibitor, etc.) or animal derived trypsin inhibitor (trypsin inhibitor from chicken or turkey egg white, from bovine pancreas, etc).

The compositions may also include an adjuvant. A number of adjuvants are well known to one skilled in the art. Suitable adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The concentration of immunogenic peptides of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990). Both of these references are incorporated herein by reference in their entirety.

Transdermal delivery systems include patches, gels, tapes and creams, and can contain excipients such as solubilizers, permeation enhancers (e.g. fatty acids, fatty acid esters, fatty alcohols and amino acids), hydrophilic polymers (e.g. polycarbophil and polyvinyl pyrollidine and adhesives and tackifiers (e.g. polyisobutylenes, silicone-based adhesives, acrylates and polybutene). Transmucosal delivery systems include patches, tablets, suppositories, pessaries, gels, and creams, and can contain excipients such as solubilizers and enhancers (e.g. propylene glycol, bile salts and amino acids), and other vehicles (e.g. polyethylene glycol, fatty acid esters and derivatives, and hydrophilic polymers such as hydroxypropylmethyl cellulose and hyaluronic acid). Injectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone. Other delivery systems that can be used for administering the pharmaceutical composition of the invention include intranasal delivery systems such as sprays and powders, sublingual delivery systems and systems for delivery by inhalation. For administration by inhalation, the pharmaceutical compositions of the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the peptides of the invention and a suitable powder base such as lactose or starch. The pharmaceutical compositions of the invention may be further formulated for administration by inhalation as e.g. described in U.S. Pat. No. 6,358,530.

In another aspect the invention relates to a method for producing a pharmaceutical composition comprising the proteins and/or peptides of the invention. The method comprises at least the steps of mixing the proteins and/or peptides of the invention with a pharmaceutically acceptable carrier and further constituents like adjuvant as described above.

Also provided is a method for the treatment or prevention of inflammatory bowel diseases (IBD) in a human, comprising administering to a person in need thereof a therapeutically or prophylactically effective amount of a protein and/or peptide as described above. In a preferred embodiment the administration is orally, at a regular interval.

It is noted that the therapeutic and prophylactic (protective) treatments described herein are not limited to the complete abolishment or complete prevention of disease, but in one embodiment also refer to a significant reduction in severity of one or more IBD symptoms in the treated subject group compared to the control group, as described in the examples. For example one or more symptoms associated with the IBD, such as the weight loss (clinical state), colon shortening, and/or histomorphological changes etc. may be significantly reduced in the treated group. A significant reduction should be statistically significant, and the skilled person can easily determine whether this is the case. For example, a reduction in one or more symptoms by at least about 1%, 2%, 5%, 10%, 20% or more, compared to the control group, may be significant.

The following non-limiting Examples describe the protective and therapeutic use of antigenic proteins and peptides of the invention. Unless stated otherwise in the Examples, all molecular techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK).

Sequences

SEQ ID NO 1: amino acid sequence of HSP60 protein of Mycobacterium tuberculosis

SEQ ID NO 2: fragment of HSP60 protein of Mycobacterium tuberculosis

SEQ ID NO 3: human HSP10 (P61604)

SEQ ID NO 4: human HSP40 (P25685)

SEQ ID NO 5: human HSP40 (Q86TL9)

SEQ ID NO 6: human HSP60 (P10809)

SEQ ID NO 7: human HSP70 (P11021)

SEQ ID NO 8: human HSP90 (P08238)

SEQ ID NO 9: human HSP100 (O76031)

### FIGURE LEGENDS

FIG. 1—shows the protective effect of HSP60, in significantly reducing the clinical score in HSP60 treated mice (Example 2).

FIG. 2—shows the protective effect of HSP60, in significantly reducing colon shortening in HSP60 treated mice (Example 2).

FIG. 3—shows the protective effect of HSP60, in significantly reducing histomorphological changes in HSP60 treated mice (Example 2).

FIG. 4—shows the protective effect of HSP60, in significantly reducing colon shortening in HSP60 treated mice (Example 3).

FIG. 5—shows the protective effect of HSP60, in significantly reducing weight loss in HSP60 treated mice (Example 3).

FIG. 6—shows the protective effect of HSP60, in significantly reducing the colitis activity index in HSP60 treated mice (Example 3).

FIG. 7—shows the protective effect of HSP60, in significantly reducing histological patterns of inflammation in HSP60 treated mice (Example 3).

FIG. 8—shows the experimental schedule used to test the therapeutic effect of HSP60

FIG. 9—shows the therapeutic effect of HSP60, in significantly reducing colon shortening in HSP60 treated mice (Example 4).

FIG. 10—shows weight loss of HSP60 treated mice compared to controls (Example 4).

FIG. 11—shows a comparison of the clinical state of HSP60 treated mice compared to controls (Example 4)

FIG. 12—shows a comparison of the histological grade of HSP60 treated mice compared to controls (Example 4)

### EXAMPLES

### Example 1

### The DSS Model of Colitis

A model of colitis that is at least partially related to a change in epithelial cell barrier function is the colitis induced by the physical agent, dextran sulfate sodium (DSS). This model has been frequently used to study the efficacy of potential therapeutic agents because of its ease to induce via administration of DSS in drinking water and because DSS induces a consistent level of colitis with a defined onset. The mechanisms of inflammation in this form of colitis are, at least initially, the activation of nonlymphoid cells such as macrophages and the release of pro-inflammatory cytokines. Changes in epithelial barrier function can be found early (several days before the onset of frank inflammation) and thus may set the stage for macrophage activation.

In the acute stages of DSS colitis the T cell response consists of a polarized Th1 response, but in later and more chronic phases of the inflammation, a mixed Th1/Th2 response occurs. In either case, DSS elicits the secretion of large amounts of TNF-α and IL-6, which are mainly responsible for the tissue damage in the disease.

### Example 2

### Protective Effect of HSP60 on DSS Colitis in Mice

In this experiment, the inventors investigated the protective effect of M. tuberculosis HSP60 in dextran sulfate sodium (DSS)-induced murine colitis. HSP60 (33 microgram) (SEQ ID NO: 1) was applied orally using gavage once a week during 4 weeks and colitis was induced 9 days later by DSS in drinking water ad libitum for 7 days. Evaluation of the colitis severity was performed by clinical state, colon length and severity of histomorphological changes in the colon mucosa.

The experimental schedule involved:
Day 0: first dose of HSP60 composition
Day 7: second dose of HSP60 composition
Day 14: third dose of HSP60 composition
Day 21: fourth dose of HSP60 composition
Day 28-Day 35: DSS

Group 1 (Placebo):
mice: 10×BALB/c female
age: 15-21 weeks
treatment: PBS 100 microlitre intragastrically 4 times (experimental day 0, 7, 14 and 21)
DSS: Experimental day 28-35 (7 days)
killed: Experimental day 35

Group 2 (HSP60):
mice: 5×BALB/c female
age: 15-21 weeks
treatment: Mycobacterial HSP60 in 100 microlitre of PBS intragastrically 4 times (experimental day 0., 7., 14. and 21) in the concentration of 0.33 mg/ml (33 μg/mouse)
DSS: Experimental day 28-35 (7 days)
killed: Experimental day 35
   Results:

Clinical State

TABLE 1
Clinical state scoring:

| Parameter | | Score |
|---|---|---|
| Weight loss | 1-10% | 1 |
| | 11-20% | 2 |
| | >20% | 3 |
| Piloerection | Puffy coat | 1 |
| Stool consistency | soft | 1 |
| | diarrhea | 2 |
| | blood in stool | 3 |

Total score is calculated by adding the score for each parameter.

TABLE 2
Results

| | PBS | HSP60 |
|---|---|---|
| Number of mice | 10 | 5 |
| Average | 6.9 | 2.8 |
| Variance | 0.32 | 4.2 |
| Standard deviation | 0.56 | 2.049 |
| Median | 7.0 | 2.0 |

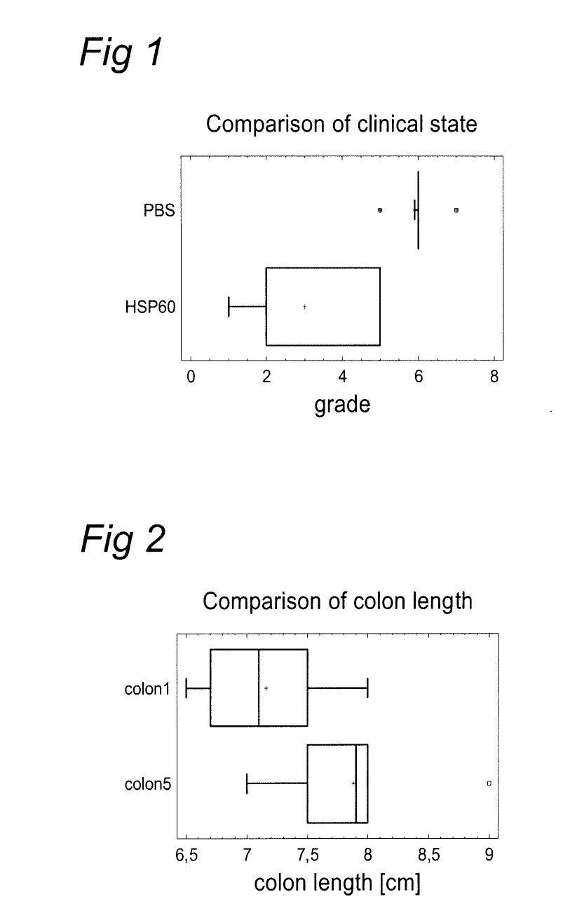

As shown in FIG. 1, mice treated with HSP60 had significant reductions in clinical score (P=0.0000389) compared with PBS-treated mice.

Colon Length

TABLE 3
Colon length results

| | PBS | HSP60 |
|---|---|---|
| Number of mice | 10 | 5 |
| Average | 7.16 | 7.88 |
| Variance | 0.242 | 0.547 |
| Standard deviation | 0.492 | 0.739 |
| Median | 7.1 | 7.9 |

As shown in FIG. 2, mice treated with HSP60 had significant reductions in colon shortening (P=0.041) compared with PBS-treated mice. Colon shortening is a sensitive marker of the inflammatory process in the colon.

Histological Grade

TABLE 4
Histological grade results

| | PBS | HSP60 |
|---|---|---|
| Number of mice | 10 | 5 |
| Average | 1.125 | 0.775 |
| Variance | 0.065 | 0.104 |
| Standard deviation | 0.256 | 0.323 |
| Median | 1.062 | 0.625 |

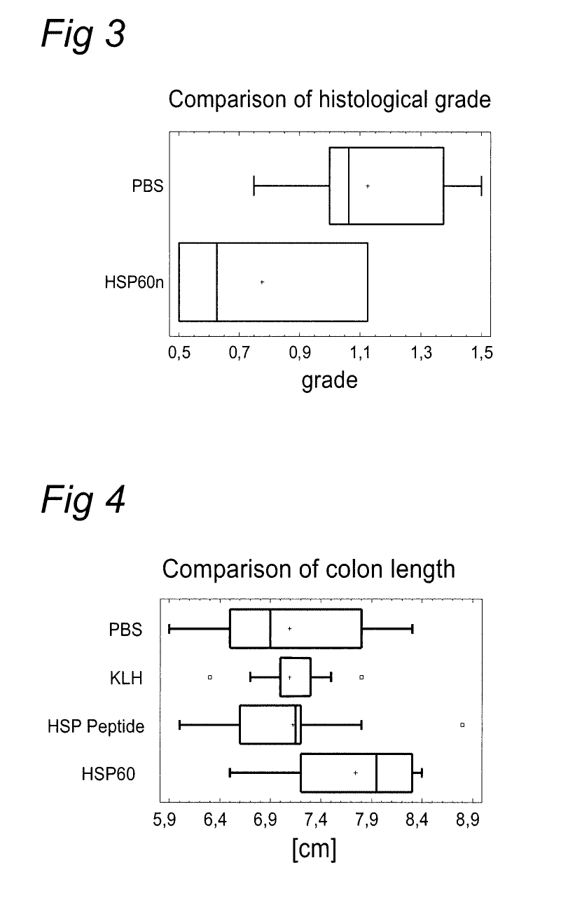

As shown in FIG. 3, mice treated with HSP60 had significant reductions in histomorphological changes in the colon mucosa (histological grading) (P=0.04) compared with PBS-treated mice.

### Example 3

### Protective Effect of HSP60 and HSP60 Peptide on DSS Colitis in BALb/c Mice

In this experiment a defined HSP60 derived peptide (SEQ ID NO: 2) was tested besides the M. tuberculosis HSP60 protein (SEQ ID NO: 1). For control purposes a KLH protein was added. The proteins and peptide (30 microgram) were applied orally using gavage once a week during 4 weeks and colitis was induced 7 days later by DSS in drinking water ad libitum for 7 days. Evaluation of the colitis severity was performed by clinical state, colon length and severity of histomorphological changes in the colon mucosa.

Group 1 (PBS):
mice: 10×BALB/c female
age: 105 days and 135 days
treated: 100 microlitre of sterile PBS intragastrically 4 times
DSS: 8 days ad libitum

Group 2 (KLH, Sigma H-2133):
mice: 10×BALB/c female
age: 105 days and 135 days
treated: KLH 30 μg/mouse in 100 μl of sterile PBS intragastrically 4 times
DSS: 8 days ad libitum

Group 3 (HSP60 peptide SEQ ID NO: 2):
mice: 10×BALB/c female
age: 105 days and 141 days
treated: HSP60 peptide 30 μg/mouse in 100 μl of sterile PBS intragastrically 4 times
DSS: 8 days ad libitum

Group 4 (HSP60):
mice: 10×BALB/c female
age: 101 days and 135 days
treated: HSP60 30 μg/mouse in 100 μl of sterile PBS intragastrically 4 times
DSS: 8 days ad libitum
   Results
   Colon Length

As shown in FIG. 4 the colon length analysis showed a significant reduction of colon shortening in the HSP60 treated mice as compared to the PBS or KLH treated mice. There was no significant effect of the HSP peptide treatment.

Weight Loss

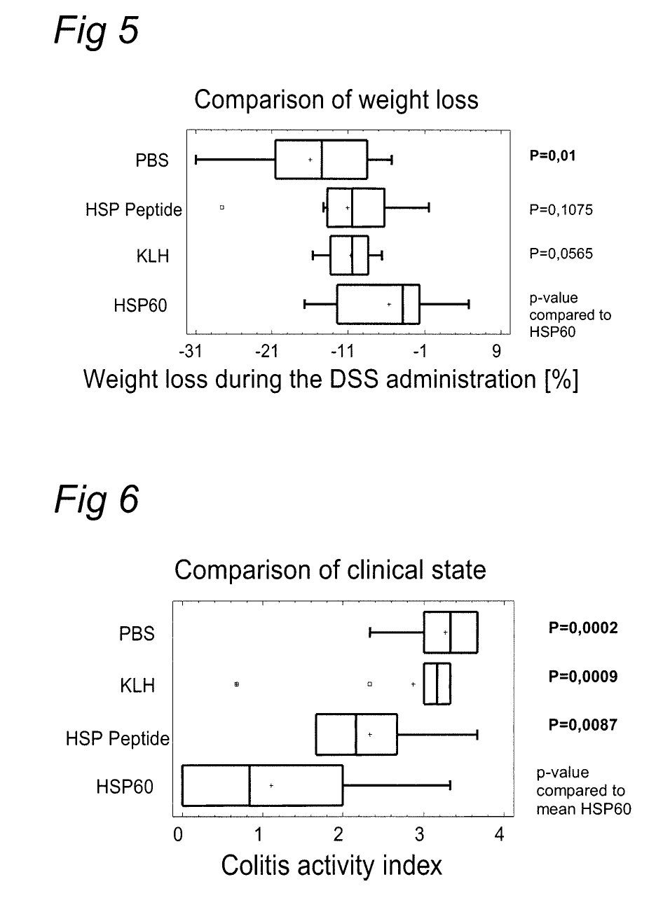

FIG. 5 shows that a significantly reduced weight loss was observed in the HSP60 treated group as compared to the PBS controls.

Clinical State

TABLE 5
results

| Parameter* | | Score |
|---|---|---|
| Weight loss | >5% | 0 |
| | 5-10% | 2 |
| | 10-20% | 3 |
| | >20% | 4 |
| Stool | well formed pellets | 0 |
| consistency | pasty and semiformed stools that | 2 |
| | don't stick to the anus | |
| | liquid stools that did stick to the anus | 4 |
| Bleeding | no blood in hemoccult | 0 |
| | positive hemoccult (Okult viditest | 2 |
| | rapid, Vidia s.r.o.) | |
| | gross bleeding | 4 |

Total score calculated by adding each parameter score and divided by 3.

Cooper et al.: Clinicopathologic study of dextran sulfate sodium experimental murine colitis. Lab Invest 69:238-249, 1993.
Hermann et al.: Specific Type IV Phosphodiesterase Inhibitor Rolipram Mitigates Experimental Colitis in Mice JPET 292:22-30, 2000

As seen in FIG. 6, the colitis activity index showed a reduction of activity in the HSP60 treated group which was significant as compared to HSP peptide, KLH and PBS controls. The colitis activity index of the HSP peptide treated group was also significantly reduced as compared to the PBS controls (p<0.002).

Colitis Activity Index

The following parameters were assessed:
Blood in the colon (inspection)
Occult Bleeding in the stool (Okult viditest rapid, Vidia s.r.o.)—If the bleeding was clearly visible, the test has not been performed and the sample was considered positive.
Visible rectal bleeding

Any gross sign of disease (diarrhoea, rectal bleeding, rectal prolaps)

TABLE 6

| | Blood | | Visible | |
|---|---|---|---|---|
| | in the | Occult | rectal | Any gross sign |
| | colon | Bleeding | bleeding | of disease |
| PBS | 6/10 | 10/10 | 9/10 | 10/10 |
| KLH | 5/10 | 9/10 | 8/10 | 9/10 |
| HSP | 4/10 | 6/10 | 3/10 | 9/10 |
| Peptide | | | | |
| HSP 60 | 2/10 | 2/10 | 2/10 | 5/10 |

Histological Grade

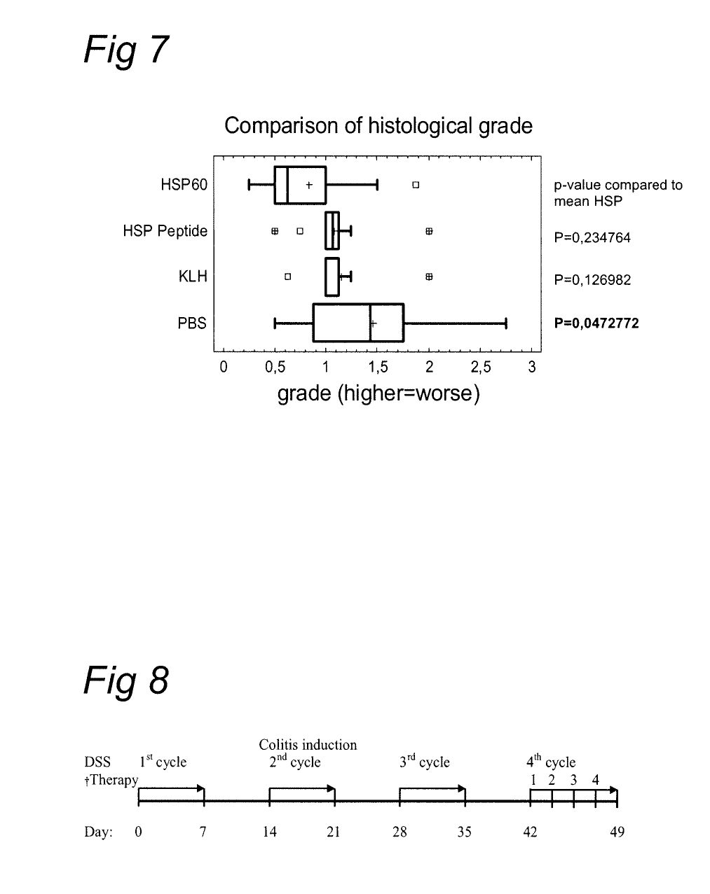

As seen in FIG. 7, the comparison of histological grades showed a reduced histological pattern of inflammation in the HSP60 group as compared to the PBS controls.

### Example 4

### Therapeutic Effect of HSP60 on DSS Colitis in BALb/c Mice

Chronic colitis was established after 4 cycles of 3% dextran sodium sulfate (dissolved in drinking water and autoclaved) administration. Each cycle took 7 days followed by 7 days without DSS.

In this experiment the HSP interventions (4 times 30 micrograms HSP or KLH in 50 microliter PBS) were started during the final (4^{th}) DSS administration. This is a protocol designed to study therapy, whereas the experiments above were targeted to prevention of disease. Evaluation of the colitis severity was performed by clinical state, colon length and severity of histomorphological changes in the colon mucosa.

In the treatments below, 50 μl of “drug” was coadministered with 1 mg of soybean trypsin inhibitor (SBTI, SIGMA T9003) dissolved in 50 μl of 0.15 mol/l sodium bicarbonate buffer and was administered intragastrically 4 times during the 4^{th} cycle (42, 44, 46 and 48^{th} day).

Group 1 (PBS):
Mice: 6×BALB/c female
Age: between 67 days and 82 days
DSS: 4 cycles of 7 days ad libitum DDS in drinking water followed by 7 days without DDS
Treated “drug”: 50 μl of sterile PBS

Group 2 (KLH, Sigma H-2133):
Mice: 6×BALB/c female
Age: between 67 days and 82 days
DSS: 4 cycles of 7 days ad libitum DDS in drinking water followed by 7 days without DDS
Treated “drug”: 30 microgram of KLH in 50 μl of sterile PBS

Group 3 (HSP60, SEQ ID NO: 1):
Mice: 6×BALB/c female
Age: between 67 days and 82 days
DSS: 4 cycles of 7 days ad libitum DDS in drinking water followed by 7 days without DDS
Treated “drug”: 30 microgram of HSP60 (SEQ ID NO: 1) in 50 μl of sterile PBS

The experiment schedule is shown in FIG. 8.

Results

Colon Length

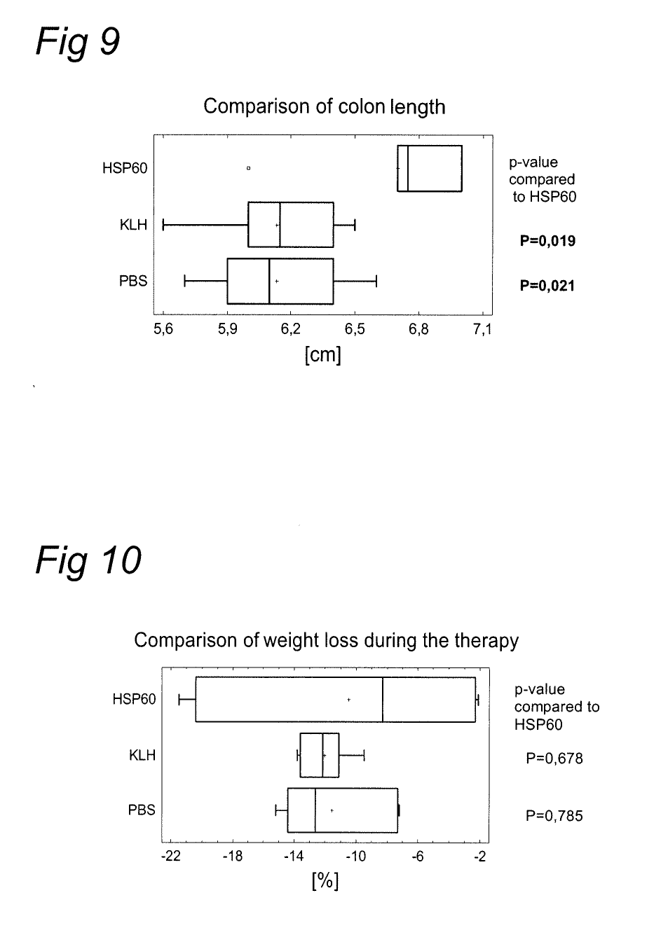

FIG. 9 shows that colon length shortening was reduced in the HSP60 treated mice.

TABLE 7
Colon length data statistical data:

| | Number | | | Standard | | | |
|---|---|---|---|---|---|---|---|
| | of mice | Average | Variance | deviation | Minimum | Maximum | Range |
| PBS | 6 | 6.13333 | 0.122667 | 0.350238 | 5.7 | 6.6 | 0.9 |
| KLH | 6 | 6.13333 | 0.110667 | 0.332666 | 5.6 | 6.5 | 0.9 |
| HSP60 | 6 | 6.7 | 0.136 | 0.368782 | 6.0 | 7.0 | 1.0 |
| Total | 18 | 6.32222 | 0.184183 | 0.429165 | 5.6 | 7.0 | 1.4 |

TABLE 8
Multiple Range Tests

| Contrast | Difference | p-value | +/−Limits |
|---|---|---|---|
| HSP60 - KLH | *0.566667 | 0.0189635 | 0.431781 |
| HSP60 - PBS | *0.566667 | 0.0212231 | 0.431781 |
| PBS - KLH | 0.0 | 1 | 0.431781 |

*Denotes a statistically significant difference.

HSP60 is protective against the chronic colitis compared to KLH or PBS.

Weight Loss (During the Treatment)

FIG. 10 showed no significant effect, but weight loss is known to be an unreliable marker of severity in chronic colitis.

Clinical State

TABLE 9

| Parameter* | | Score |
|---|---|---|
| Weight loss | >5% | 0 |
| | 5-10% | 2 |
| | 10-20% | 3 |
| | >20% | 4 |
| Stool | Well formed pellets | 0 |
| consistency | Pasty and semiformed stools that | 2 |
| | don't stick to the anus | |
| | Liquid stools that did stick to the anus | 4 |
| Bleeding | No blood in hemoccult | 0 |
| | Positive hemoccult (Okult viditest rapid, | 2 |
| | Vidia s.r.o.) | |
| | Gross bleeding | 4 |

Total score is calculated by adding the score for each parameter and divided by 3.
*Cooper et al.: Clinicopathologic study of dextran sulfate sodium experimental murine colitis. Lab Invest 69: 238-249, 1993.
*Hermann et al.: Specific Type IV Phosphodiesterase Inhibitor Rolipram Mitigates Experimental Colitis in Mice JPET 292: 22-30, 2000

TABLE 10
Summary Statistics

| | Number | | | Standard | | | |
|---|---|---|---|---|---|---|---|
| | of mice | Average | Variance | deviation | Minimum | Maximum | Range |
| PBS | 6 | 2.667 | 0.000000 | 0.000000 | 2.667 | 2.667 | 0 |
| KLH | 6 | 3.33333 | 0.133333 | 0.365149 | 2.667 | 3.667 | 1.0 |
| HSP60 | 6 | 2.0555 | 0.907993 | 0.952887 | 1.333 | 3.667 | 2.334 |
| Total | 18 | 2.68533 | 0.594599 | 0.771102 | 1.333 | 3.667 | 2.334 |

TABLE 11
Multiple Range Tests

| Contrast | Difference | p-value | +/−Limits |
|---|---|---|---|
| HSP60 - KLH | *−1.27783 | 0.0118954 | 0.725017 |
| HSP60 - PBS | −0.6115 | 0.146944 | 0.725017 |
| PBS - KLH | *0.666333 | 0.00119969 | 0.725017 |

*Denotes a statistically significant difference

HSP60 is protective against colitis compared to KLH. (As well as the PBS seems to be protective compared to KLH). Problematic category “weight loss” is included in the score.

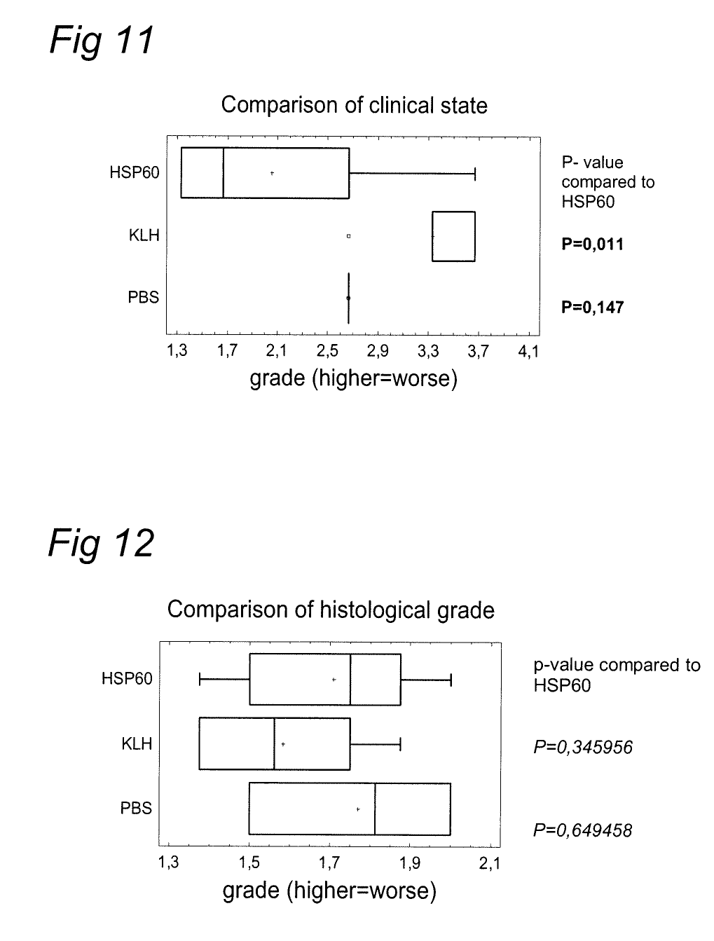

FIG. 11 shows that, for the clinical status, HSP60 protected from severe colitis compared to KLH. Problematic category “weight loss” was included in the clinical score.

Histological Grade

TABLE 12
Summary statistics

| | Number | | | Standard | | | |
|---|---|---|---|---|---|---|---|
| | of mice | Average | Variance | deviation | Minimum | Maximum | Range |
| PBS | 6 | 1.70833 | 0.0541667 | 0.232737 | 1.375 | 2.0 | 0.625 |
| KLH | 6 | 1.58333 | 0.0416667 | 0.204124 | 1.375 | 1.875 | 0.5 |
| HSP60 | 6 | 1.77083 | 0.0526042 | 0.229356 | 1.5 | 2.0 | 0.5 |
| Total | 18 | 1.6875 | 0.0500919 | 0.223812 | 1.375 | 2.0 | 0.625 |

TABLE 13
Multiple Range Tests

| Contrast | Difference | p-value | +/−Limits |
|---|---|---|---|
| HSP60 - KLH | 0.125 | 0.345956 | 0.273733 |
| HSP60 - PBS | −0.0625 | 0.649458 | 0.273733 |
| PBS - KLH | −0.1875 | 0.165568 | 0.273733 |

FIG. 12 shows the result of this test.

1. A method for the treatment or prevention of inflammatory bowel disease (IBD), comprising administering to a mammal in need thereof an effective amount of a full length heat shock protein or a polypeptide at least 98% identical in sequence thereto.

2. The method according to claim 1, wherein the full length heat shock protein is a bacterial or mammalian.

3. The method according to claim 2, wherein the mammalian full length heat shock protein is human.

4. The method according to claim 1, wherein the IBD is lymphocyte colitis, collagenous colitis, coeliac disease, Crohn's disease or ulcerative colitis.

5. The method according to claim 1, wherein said full length heat shock protein comprises at least 35% amino acid sequence identity over the entire length to a mammalian heat shock protein selected from the group consisting of HSP10, HSP40, HSP60, HSP70, HSP90 and HSP100.

6. The method according to claim 2, wherein the bacterial heat shock protein is derived from a bacterium belonging to a genus Mycobacterium, Norcadia, Nocardioides, Cornybacterium, Arthorbacter, Propionibacterium, Brevibacterium, Kineococcus, Streptomyces, Thermobifida, Bifidobacterium, Rubrobacter, Synechococcus, Heliobacillus, Clostridium, Bacillus, Staphylococcus, Streptococcus, Enterococcus, Coxellia, Mesorhizobium, Lactobacillus or Lactococcus.

7. The method according to claim 1, wherein the heat shock protein is a HSP60 protein.

8. The method according to claim 1, wherein said full length protein comprises the amino acid sequence of SEQ ID NO: 1 or the amino acid sequence of SEQ ID NO: 6.

9. The method according claim 1, in which the administering is oral, nasal, transdermal, parenteral or inhalation.

10. The method according to claim 1, in which the effective amount is at least 0.1 μg of the protein/kg body weight of the mammal.

11. A method for the treatment or prevention of inflammatory bowel disease (IBD), comprising administering to a mammal in need thereof an effective amount of an isolated peptide consisting essentially of a fragment of at least 6 contiguous amino acids of a heat shock protein.

12. The method according to claim 11, wherein the fragment consists of the amino acid sequence of SEQ ID NO: 2.

13. The method according to claim 11, wherein the fragment is selected from the group consisting of: amino acids 84-95, 171-175, 247-252, 254-261, 253 -268 272-281, 365-370 and 403-410, each of SEQ ID NO: 1.

14. The method according to claim 11, wherein the fragment is at least 15 contiguous amino acids of a heat shock protein.

15. The method according to claim 11, wherein the heat shock protein consists of the amino acid sequence of SEQ ID NO: 1, the amino acid sequence of SEQ ID NO: 6, or a protein having at least 98% amino acid sequence identity thereto.

16. A pharmaceutical composition for the treatment or prevention of IBD comprising an effective amount of a protein of SEQ ID NO: 1, SEQ ID NO: 6, or a protein having at least 98% amino acid sequence identity with either sequence.

17. The pharmaceutical composition according to claim 16, in which the composition is in oral dosage form.

18. The method according to claim 16, in which the oral dosage form comprises at least 7.5 μg/dose of the protein.

19. A pharmaceutical composition for the treatment or prevention of IBD comprising an effective amount of an isolated peptide consisting essentially of a fragment of at least 6 contiguous amino acids of a heat shock protein of SEQ ID NO: 1 or SEQ ID NO: 6, or a protein comprising at least 98% amino acid sequence identity with either sequence.

20. The pharmaceutical composition according to claim 19, wherein the fragment is at least 15 amino acids in length.

21. The pharmaceutical composition according to claim 19, wherein the fragment is 6-16 amino acids in lengths.

22. The pharmaceutical composition according to claim 19, wherein the fragment is 10-20 amino acids in lengths.

23. The pharmaceutical composition according to claim 19, wherein said protein fragment consists of the amino acid sequence of SEQ ID NO: 2 or at least one amino acid fragment selected from the group consisting of: amino acids 84-95, 171-175, 247-252, 254-261, 253 -268 272-281, 365-370 and 403-410, each of SEQ ID NO: 1.
